(19) United States
(12) Patent Application Publication
(10) Pub. No.: US 2011/0196204 A1
(43) Pub. Date: Aug. 11, 2011

(54) Shape-conforming intubation device

(75) Inventors: Harsha Setty, Martinez, GA (US); Richard Schwartz, Evans, GA (US); John Schwartz, Williamston, MI (US)

(73) Assignee: Al Medical Devices, Inc., Williamston, MI (US)

(21) Appl. No.: 12/931,746

(22) Filed: Feb. 9, 2011

Related U.S. Application Data
(60) Provisional application No. 61/337,678, filed on Feb. 11, 2010.
(60) Provisional application No. 61/456,590, filed on Nov. 9, 2010.

(51) Int. Cl. A61M 16/04; A61B 1/04; A61B 1/06

(52) U.S. Cl. 600/120; 128/200.26

(57) ABSTRACT

The disclosure generally relates to an endotracheal intubation device 100 including a channel element 120 that defines a first channel 122 and additional retaining structure that defines a second channel 140. The device 100 is reversibly movable between a first relaxed position A (e.g., a generally extended or straight position) and a second curved position B (e.g., an articulated, generally non-linear position) with an articulating means 160. An endotracheal tube 200 can be inserted into the second channel 140, and the intubation device 100 then can be used to intubate a patient P according to a disclosed intubation method.

### CROSS-REFERENCE TO RELATED APPLICATIONS

Priority is claimed to U.S. Provisional Application Nos. 61/337,678, filed on Feb. 11, 2010, and 61/456,590, filed on Nov. 9, 2010, the disclosures of which are incorporated herein in their entireties.

### FIELD OF THE DISCLOSURE

The disclosure relates to endotracheal intubation devices, particularly to endotracheal devices having an improved means for curving the distal end and guiding of an endotracheal tube inserted into a patient.

### BRIEF DESCRIPTION OF RELATED TECHNOLOGY

U.S. Pat. No. 4,086,919 to Bullard describes a laryngoscope that permits indirect visualization of the glottis. This device reduced the need to move the head of the patient from the neutral position. The distal end of the longitudinal axis was also fixed.

U.S. Pat. No. 5,645,519 to Lee describes an endoscopic instrument with a tubular member passed alongside a blade. It also possesses a viewing device at the proximal end for viewing placement of an endotracheal tube. The device itself is concentric with the endotracheal tube. The track means are aligned in preset orientations.

U.S. Pat. No. 4,611,579 to Bellhouse describes an angled laryngoscope blade with a planar component. The angular portion of the blade is fixed but allows for navigation around abnormalities.

U.S. Pat. No. 6,843,769 to Gandarias discloses a hollow laryngoscope with a fixed anatomical curve that incorporated a channel securing the endotracheal tube that ran parallel to the visualization channel. This channel allowed for removal of the device after the endotracheal tube is in place. A fixed blade at the distal end of the curved section assisted in securing tissue such as the epiglottis. The visualization channel in this embodiment consisted of mirrors.

U.S. Pat Application 2009/0198111 to Nearman describes a dynamically articulating laryngoscope blade controlled from any handle at multiple points. The control unit consists of both coarse and fine control to achieve various configurations. The curvable section consists of individual metal plates articulating relative to each other.

U.S. Pat Application 2007/0106121 to Yokota describes a rigid intubation instrument with an integrated guide means for passing an endotracheal tube through a target site. The integrated guide allows for removal of the intubation tube to enable removal of the device from the patient's mouth.

U.S. Pat. No. 4,861,153 issued to Berci discloses an intubating video endoscope which includes an elongated sheath member with a selectively controllable bendable section housing an image forming optical system. A generally rigid section includes a control housing. An image transmitting optical system extends throughout the length of the sheath member and terminates adjacent to the image forming system. A light transmitting system also extends throughout the length of the sheath member to the image forming optical system, the rearward end of which is adapted to be operatively connected to a light source.

U.S. Pat. No. 6,539,942 to Schwartz et al., hereby incorporated herein by reference in its entirety, describes an endotracheal intubation device having a series of interlinked, truncated ring-like elements disposed along the distal portion of the tube and a handgrip for controlling the degree of bend in the distal end of the device. An imaging device, such as a nasopharyngoscope, can be inserted through the intubation device to visualize the patient's vocal cords during the intubation procedure. The endotracheal intubation device uses a scissors mechanism without pulleys to bend the distal end of the device.

U.S. Pat. No. 3,802,440 issued to Salem et al. discloses an adjustably flexible intubation guide to aid in the insertion of a tubular-type device into a body duct or passage. The guide includes a flexible tube member with a rod member inserted therein. The rod member is slidable within the tube member, and the rod member and the tube member are firmly connected at the tip of the distal ends thereof.

U.S. Pat. No. 4,832,020 issued to Augustine discloses a tracheal intubation guide with a tubular member having a curved forward end shaped to follow the curvature of the back of the tongue and throat of a patient, a rear end for projecting out through the mouth of the patient, and an anterior guide surface extending along at least part of the length of the member to its forward end for guiding the member into the throat into a position opposite the opening into the larynx.

U.S. Pat. No. 4,832,020 issued to Gomez discloses an intubating assembly used to position an intubation tube into a trachea of a patient. The intubating assembly has a guide assembly that receives the intubation tube therein and conforms the intubation tube to its configuration. The guide assembly includes first and second introduction segments hingedly coupled to one another and positionable between a closed orientation, which defines a generally curved configuration of the guide assembly, and an open orientation, which defines a generally straight configuration of the guide assembly.

U.S. Pat. No. 7,458,375, U.S. Pat. No. 7,658,708, U.S. Publication No. 2008/0200761, U.S. Publication No. 2008/0308098, and U.S. Publication No. 2010/0095969 to Schwartz et al. are directed to endotracheal intubation devices having a curveable portion and internal optics or a viewing device which facilitate the insertion of an endotracheal tube into a patient.

U.S. Publication No. 2008/0208000 to Schwartz et al. is directed to a device for endotracheal intubation and fluid delivery into the trachea of a patient. The fluid delivery device includes a tubular housing adapted to be sealably mounted on an elongate element of the endoscope and delivers a fluid thereto.

U.S. Publication No. 2009/0090357 to Schwartz et al. is directed to a guide/laryngoscope blade device for facilitating the insertion of a medical device into the trachea of a patient.

### OBJECTS

While the related art discloses endotracheal intubation devices, there still exists a need for an improved endotracheal device having both a rigid and curvable section and optionally internal optics so as to facilitate both the insertion and guidance of an endotracheal tube into a patient.

Therefore, it is an object of the present disclosure to provide an improved endotracheal intubation device having a flexible portion. These and other objects will become increasingly apparent by reference to the following description.

### SUMMARY

The disclosure relates to an endotracheal intubation device comprising: (a) a channel element having a proximal end and a distal end, the channel element comprising (i) a rigid channel portion at the proximal end of the channel element and (ii) a curveable channel portion at the distal end of the channel element, the curveable channel portion being operatively connected to the rigid channel portion, wherein the rigid channel portion and the curveable channel portion together define a first channel that (A) extends from the proximal end to the distal end and (B) defines a centerline direction between the proximal end and the distal end; (b) an articulating means extending through the first channel between the proximal end and the distal end, the articulating means being operatively connected to the curveable channel portion for articulation; and (c) a second channel adjacent the first channel and extending in the centerline direction along at least a segment of the rigid channel portion and at least a segment of the curveable channel portion; wherein: (i) the curveable channel portion and the second channel are together continuously and reversibly moveable between a first relaxed position (e.g., substantially straight or slightly/less curved relative to the second curved position) and a second curved position upon actuation of the articulating means; and (ii) the second channel (A) exerts a retaining force on an endotracheal tube when present in the second channel and the second channel is in the curved position and (B) relaxes the retaining force to release the endotracheal tube as the second channel moves from the curved position toward the relaxed position. The intubation device can further comprise a protrusion extending in the centerline direction from the distal end of the curveable channel portion, the protrusion being operable to lift the epiglottis of a patient when inserted into the trachea of a patient. The channel element can be formed from stainless steel or a shape memory alloy (SMA) such as a nitinol nickel-titanium alloy.

Various refinements of the endotracheal intubation device are possible. For example, in an embodiment, (i) the curveable channel portion comprises one or more curveable channel elements (e.g., having a rectangular, circular, or other cross section); (ii) one curveable channel element is connected at its proximal end to the distal end of the rigid channel portion and the other curveable channel elements, when present, are connected at the proximal end thereof to the distal end of an adjacent curveable channel element; (iii) each curveable channel element defines a gap (e.g., wedge-shaped, slit-shaped, etc.) on a curveable side of the curveable channel portion, the gap being located between the curveable channel element and (A) the rigid channel portion, (B) an adjacent curveable channel element, or (C) both (A) and (B); and (iv) the gap changes in its extent as the curveable channel portion and the second channel move between the first relaxed position and the second curved position. In one refinement, the gap is at its maximum extent when the curveable channel portion and the second channel are in the first relaxed position, and the gap is closed or at its minimum extent when the curveable channel portion and the second channel are in the second curved position. In another refinement, the gap is at its minimum extent when the curveable channel portion and the second channel are in the first relaxed position, and the gap is at its maximum extent when the curveable channel portion and the second channel are in the second curved position. In another embodiment, (i) each curveable channel element is hingedly connected along a first edge of the curveable channel element to the rigid channel portion or an adjacent curveable channel element; and (ii) the gap of each curveable channel element is defined by a second, opposing edge of the curveable channel element.

In another refinement, the second channel is partially open around its circumference and along its length in the centerline direction, and the partially open structure permits access to the second channel interior other than through the proximal and distal ends of the second channel. For example, (i) the second channel can be defined by one or more retaining lips and one or more overhang structures, both of which generally extend outwardly from the first channel in a direction that is substantially normal to the centerline direction; and (ii) the retaining lips can comprise a curved surface that is sized and located to retain an endotracheal tube within the second channel. In an embodiment, the second channel extends along only a distal segment of the rigid channel portion. In another embodiment, (i) the second channel is defined by a first retaining surface and an opposing second retaining surface, each extending from or defined by a flexible sheath (e.g., a flexible polymer such as a flexible silicone polymer) encasing at least a portion of the channel element; and (ii) the first retaining surface and second retaining surface are sized and located to retain an endotracheal tube within the second channel, In the second channel, the retaining force can comprise a frictional force between an interior surface of the second channel and an exterior surface of the endotracheal tube, the diameter of the endotracheal tube being sized correspondingly to the cross sectional size of the second channel.

In another refinement, the articulating means is anchored to an interior wall of the first channel at or near the distal end. The articulating means can comprise a control wire that is anchored to an interior wall of the first channel at or near the distal end. Alternatively, the articulating means can comprise a push rod that is anchored to an interior wall of the first channel at or near the distal end.

In another refinement, the endotracheal intubation device further comprises a flexible gap cover plate on an outside wall of the channel element adjacent the gap or gaps, wherein: (i) the gap cover plate covers at least a portion of the gap or gaps and at least partially shields the first channel interior from the external environment; (ii) the gap cover plate is anchored to the channel element in one location (e.g., one of the curveable channel elements, the rigid channel portion); (iii) the gap cover plate is slidably retained at one or more locations on the channel element in the centerline direction; and (iv) the gap cover plate is flexible so that it conforms to the shape of the curveable channel portion as the curveable channel portion moves between the first relaxed position and the second curved position. The gap cover plate can be slidably retained by the curveable channel element with a retaining band thereon that defines a channel through which the gap cover plate can slide in the centerline direction. The endotracheal intubation device additionally can comprise a retaining sleeve at the distal end of the rigid channel portion, at the proximal end of the curveable channel portion, or both, wherein the retaining sleeve is positioned and sized to enclose a freely moving end of the gap cover plate as the curveable channel portion moves between the first relaxed position and the second curved position.

In another refinement, the intubation device further comprises a sensor means mounted within the first channel at or near the distal end of the curveable channel portion. The sensor means can comprise (i) an imaging unit selected from the group consisting of a CMOS imager, a CCD imager, an FPA imager, an IR imager, and an ultrasonic imager and (ii) optionally an illumination source to enhance the imaging ability of the imaging unit. Suitably, the sensor means has a sensing axis that is directed toward a placement axis extending from the distal end of the second channel in the centerline direction. The intubation device additionally can comprise a gripping means that comprises an actuating means for the articulation means, wherein: (i) the proximal end of the rigid channel portion is mounted to the gripping means; and (ii) the actuating means is operably connected to the proximal end of the articulating means. When the sensor means is included, the gripping means additionally can comprise a viewing means (e.g., LCD, OLED display) electrically connected to the sensor means through the first channel.

The disclosure also relates to a method of intubating a patient, the method comprising: (a) providing the endotracheal intubation device according to any of its various disclosed embodiments; (b) advancing an endotracheal tube through the second channel of the endotracheal intubation device; (c) inserting the distal end of the channel element with the endotracheal tube in the second channel into a patient's mouth, wherein the curveable channel portion and the second channel are in the first relaxed position during insertion; (d) actuating the articulating means to move the curveable channel portion and the second channel toward the second curved position by an amount sufficient to allow the distal end of the channel element and the endotracheal tube to be safely advanced in the throat of the patient; (e) advancing the distal end of the channel element to a position allowing guidance of the endotracheal tube into the trachea of the patient; (f) advancing the distal end of the endotracheal tube into the trachea of the patient; (g) releasing the articulating means, thereby relaxing the curveable channel portion and allowing the curveable channel portion to conform to an interior patient passageway defined by the patient's anatomy; and (h) removing the endotracheal intubation device from the patient's mouth while holding the endotracheal tube in place. In an embodiment, (i) the retaining force comprises a frictional force between an interior surface of the second channel and an exterior surface of the endotracheal tube, the diameter of the endotracheal tube being sized correspondingly to the cross sectional size of the second channel; and (ii) the release of the articulating means in part (g) reduces the frictional force, thereby facilitating the removal of the endotracheal intubation device in part (h).

The following U.S. patents and patent applications are incorporated by reference herein in their entireties for all purposes: Ser. No. 11/230,392 (filed Sep. 29, 2005 now U.S. Pat. No. 7,658,708), Ser. No. 11/514,486 (filed Sep. 1, 2006; now U.S. Pat. No. 7,458,375), Ser. No. 11/820,117 (filed Jun. 18, 2007; now U.S. Publication No. 2008/0308098), Ser. No. 11/906,870 (filed Oct. 4, 2007; now U.S. Publication No. 2009/0090357), Ser. No. 12/148,033 (filed Apr. 16, 2008; now U.S. Publication No. 2008/0208000), Ser. No. 12/148,050 (filed Apr. 16, 2008; now U.S. Publication No. 2008/0200761), Ser. No. 12/587,905 (filed Oct. 15, 2009; now U.S. Publication No. 2010/0095969), Ser. No. 12/592,406 (filed Nov. 24, 2009), Ser. No. 12/924,358 (filed Sep. 24, 2010), and Ser. No. 12/928,126 (filed Dec. 3, 2010). In general, the structure, construction, and methods for the endotracheal intubation devices disclosed herein can be incorporated into the endotracheal intubation devices of the foregoing patents/patent applications.

All patents, patent applications, government publications, government regulations, and literature references cited in this specification are hereby incorporated herein by reference in their entirety. In case of conflict, the present description, including definitions, will control.

Additional features of the disclosure may become apparent to those skilled in the art from a review of the following detailed description, taken in conjunction with the examples, drawings, and appended claims, with the understanding that the disclosure is intended to be illustrative, and is not intended to limit the claims to the specific embodiments described and illustrated herein.

### BRIEF DESCRIPTION OF THE DRAWINGS

For a more complete understanding of the disclosure, reference should be made to the following detailed description and accompanying drawings wherein:

FIG. 1 is a side view of an endotracheal intubation device according to the disclosure in a relaxed, extended position.

FIG. 2 is a top view of the endotracheal intubation device of FIG. 1.

FIG. 3 is a side view of the endotracheal intubation device in an articulated, curved position.

FIG. 4 is a side view of a channel element and a curveable channel portion of the intubation device in a partially articulated position.

FIG. 5 is a side cross sectional view of the channel element and the curveable channel portion of the intubation device in a fully articulated position.

FIG. 6 is a top, partially cutaway view of the distal end of the curveable channel portion of the intubation device.

FIG. 7 is a front view of the distal end of the curveable channel portion of the intubation device.

FIG. 8 is a side cross sectional view of the curveable channel portion of the intubation device.

FIG. 9 is a side view of the proximal end of the endotracheal intubation device mounted into a gripping means and further including an endotracheal tube inserted into the intubation device.

FIG. 10 is a top view of the endotracheal intubation device, endotracheal tube, and gripping means of FIG. 9.

FIG. 11 is a side view of the endotracheal intubation device, endotracheal tube, and gripping means of FIG. 9 illustrating the actuation of the articulating means and the corresponding movement between the extended and curved positions of the curveable channel portion.

FIG. 12 is a side cross sectional view of the channel element and the curveable channel portion of the intubation device in a fully articulated position in an alternate embodiment using a push rod as an articulating means.

FIG. 13 is a side cross sectional view of the gripping means operably connected to the push rod articulating means.

FIG. 14 illustrates a method of intubating a patient with an endotracheal tube using an endotracheal intubation device according to the disclosure.

FIG. 15 is a perspective side view of an endotracheal intubation device according to an alternate embodiment of the disclosure in a relaxed (yet partially curved), extended position.

FIG. 16 is a perspective side view of the endotracheal intubation device of FIG. 15 in an articulated, curved position.

FIG. 17 is a front cross sectional view of a flexible sheath for the endotracheal intubation device (A: without the endotracheal intubation device inserted therein; B: with the endotracheal intubation device inserted therein).

FIG. 18 is a top view of the distal end of the flexible sheath of FIG. 17.

While the disclosed apparatus and methods are susceptible of embodiments in various forms, specific embodiments of the disclosure are illustrated in the drawings (and will hereafter be described) with the understanding that the disclosure is intended to be illustrative, and is not intended to limit the claims to the specific embodiments described and illustrated herein.

### DETAILED DESCRIPTION

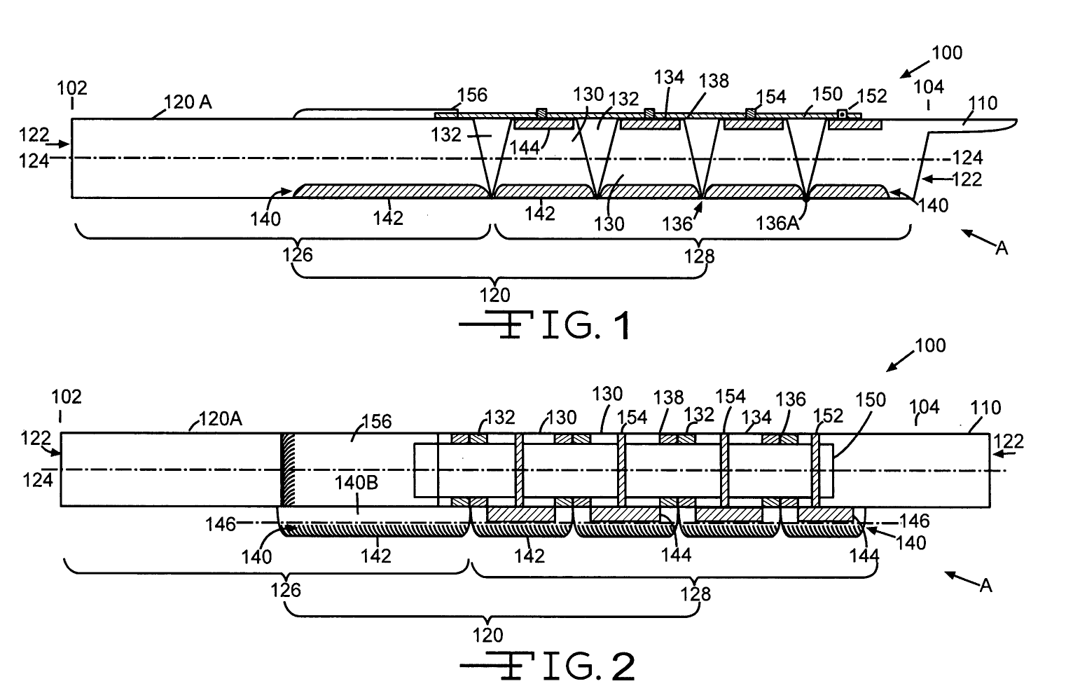

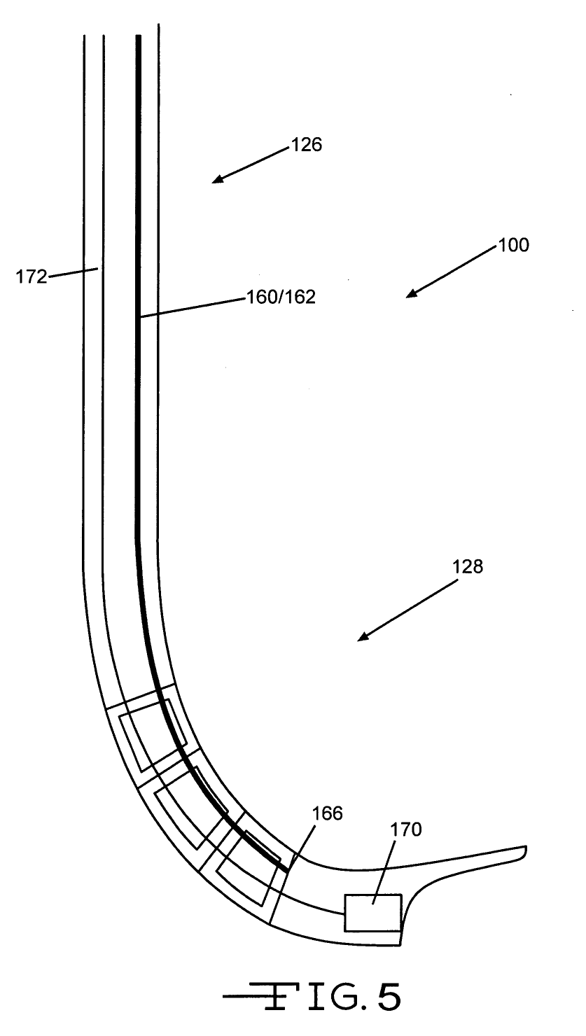

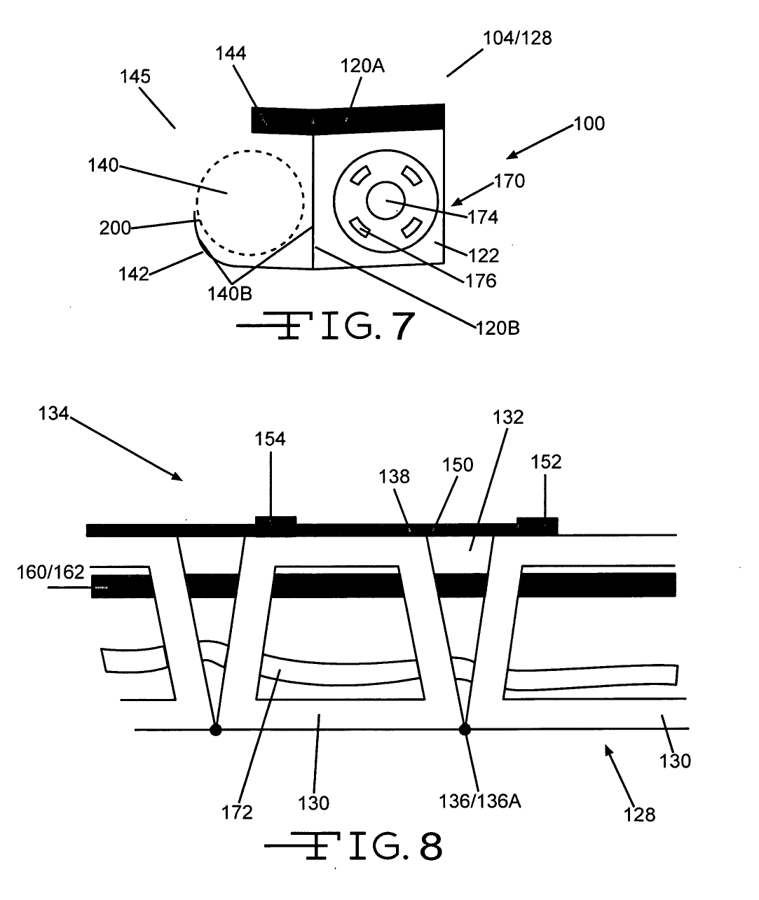

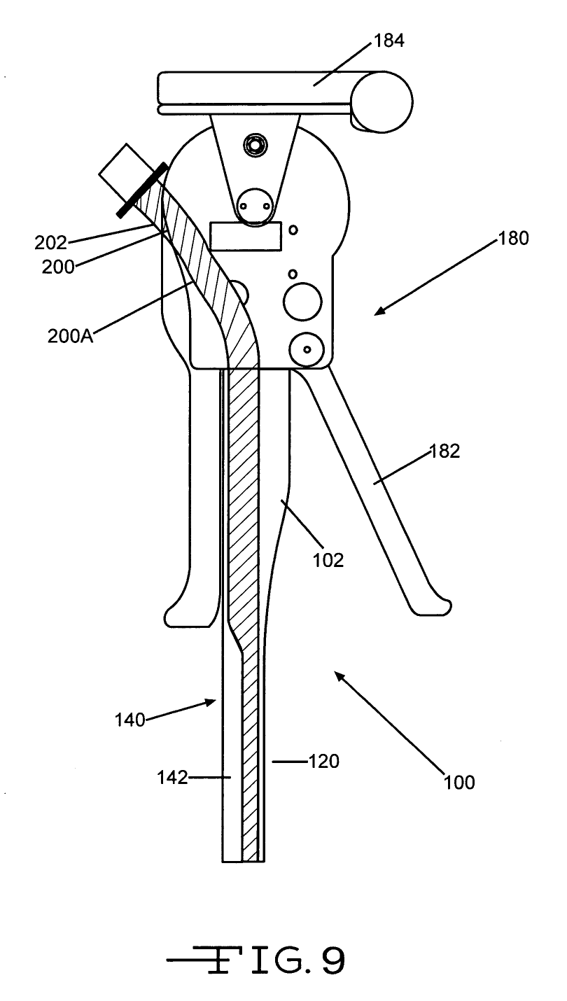

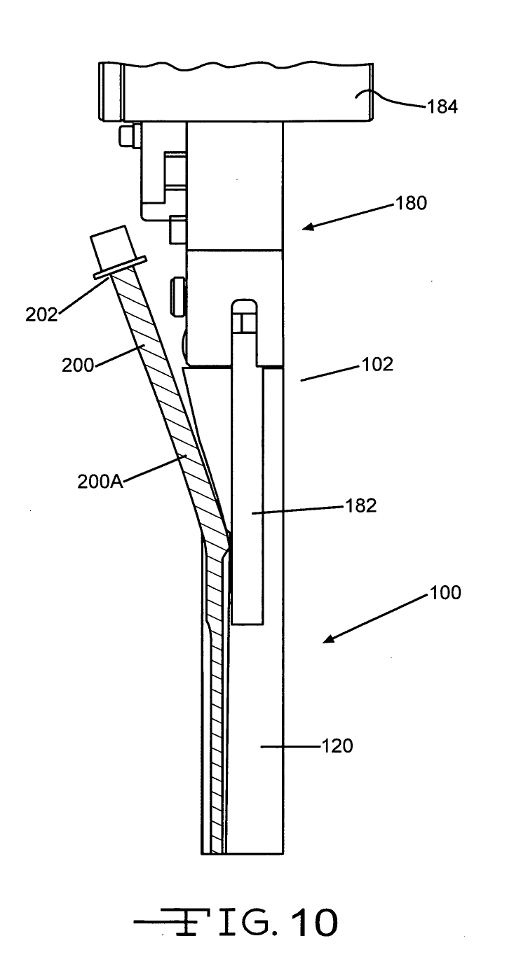

With reference to FIGS. 1-18, the present disclosure generally relates to an endotracheal intubation device 100 including a channel element 120 that defines a first channel 122 and additional retaining structure that defines a second channel 140. The device 100 is reversibly movable between a first relaxed position A (e.g., a generally extended or straight position) and a second curved position B (e.g., an articulated, generally non-linear position) with an articulating means 160. An endotracheal tube 200 can be inserted into the second channel 140, and the intubation device 100 then can be used to intubate a patient P, for example according to the intubation method disclosed herein.

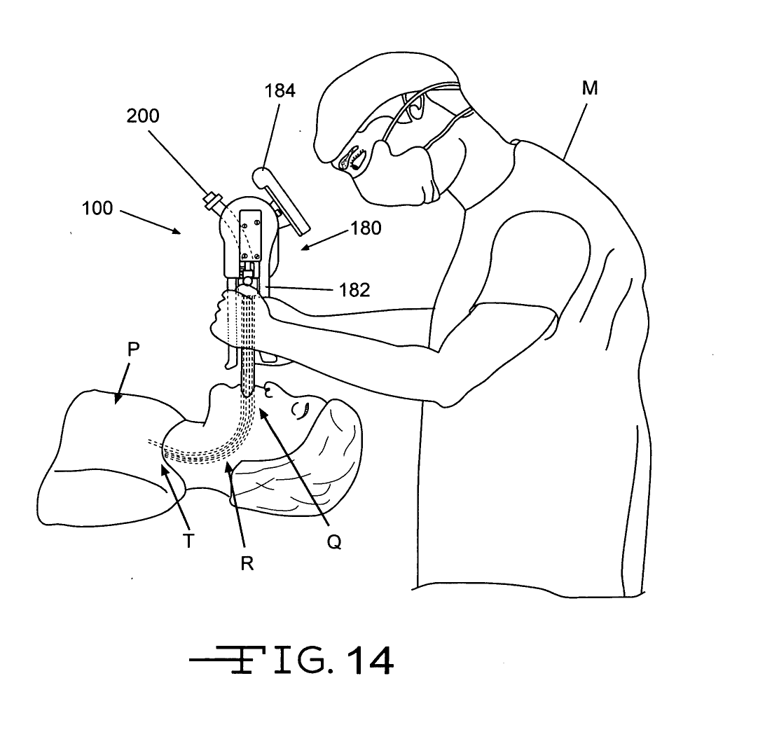

FIG. 14 illustrates the endotracheal intubation device 100 in any of its various disclosed embodiments in use on a patient P. The device 100 is operated by a physician or medical professional M to access the patient P's trachea T by inserting the endotracheal tube 200 that is mounted in the second channel 140 of the device 100 into the patient P via the mouth. The endotracheal tube 200 is constructed from a generally flexible material (e.g., silicone) so that it can deform along with the intubation device 100 when the articulating means 160 is actuated and it can maintain a shape that conforms to the tracheal passageways of the patient P upon removal of the intubation device 100. Prior to insertion, the endotracheal tube 200 is advanced through/into the second channel 140 of the intubation device 100 (e.g., by threading the tube 200 through open ends of the second channel 140 or by press-fitting a flexible tube 200 through a circumferential gap in the second channel 140), for example such that a distal end 204 of the tube 200 is near the distal end 104 of the channel element 120.

The distal end 104 of the channel element 120 is then inserted into the mouth of the patient P, along with the distal end 204 of endotracheal tube 200. At this point, the curveable channel portion 128 and the second channel 140 generally are in the first relaxed position A, although the distal end 104 of the intubation device 100 can move somewhat to accommodate the internal passageways of the patient P. Upon insertion into the patient P, the articulating means 160 is actuated to deform the channel element 120/first channel 122 and allow insertion of the intubation device 100 and the endotracheal tube 200 through curved passageways of the patient P (e.g., mouth, throat, pharynx, larynx, and/or trachea). Specifically, as the device 100 and tube 200 are inserted/advanced; the articulating means 160 is actuated to move the curveable channel portion 128, the second channel 140, and the endotracheal tube 200 (when present) together toward the second curved position B by an amount sufficient to allow the distal ends 104, 204 of the channel element 120 and the endotracheal tube 200 to be safely advanced in the throat of the patient P. By actuating the articulating means 160 to the extent necessary (e.g., by applying/releasing pressure on a gripping means 180), the distal end 104 of the channel element 120 is advanced to a position within the patient P that allows guidance of the endotracheal tube 200 into the trachea T of the patient P. For example, the distal end 104 of the channel element 120 can be advanced along with the endotracheal tube 200 (i.e., secured in place in the second channel 140) through the patient's mouth Q and into the pharynx R (e.g., oral and/or laryngeal parts thereof) to a point where it lifts/holds the patient's epiglottis (e.g., via the protrusion 110), but is above the vocal cords, larynx, and trachea T. Then, the distal end 204 of the endotracheal tube 200 can be further advanced into the trachea T of the patient P (e.g., using a sensor means 170 to remotely view the tube 200 as it is advanced while the intubation device 100 remains stationary). For example, as shown in FIG. 14, the physician M holds the intubation device 100 in place via the gripping means 180 with his/her right hand, while the left hand can be used to gradually thread/advance the tube 200 into its desired location in the trachea T.

Once the endotracheal tube 200 is in place, the articulating means 160 is released, thereby relaxing the curveable channel portion 128 and allowing the curveable channel portion 128 to conform to an interior patient passageway defined by the patient's anatomy (e.g., a curved path defined through all or a portion of the patient's mouth, pharynx, larynx, and/or trachea), for example by allowing the curveable channel portion 128 to relax/move at least partially from its curved position (i.e., which can be curved to a degree less than that of the fully articulated state B) towards the first relaxed position A. However, even when pressure is removed from the articulating means 160, the curvature of the patient's interior anatomy will maintain the curveable channel portion 128 in a partially curved state. The relaxation of the articulating means 160 and the movement towards the first position A additionally reduces the retaining force (e.g., frictional force) between the interior surface 140B of the second channel 140 and the endotracheal tube 200, thereby facilitating the removal of the endotracheal intubation device 100 from the patient P. Specifically, as shown in FIG. 14, the physician M can hold the endotracheal tube 200 in its desired location (e.g., advanced past the distal end 104 of the channel element 120 and into the trachea T) with his/her left hand, while the right hand is used to gradually pull and remove the intubation device 100 from the patient P (i.e., the tube 200 is stationary and remains in place in the patient P while the second channel 140 slides over the tube 200 as device 100 is removed). Eventually, the intubation device 100 is entirely removed from the patient P. The device 100 can be disengaged from the endotracheal tube 200, for example by pulling the device 100 and its second channel 140 past the proximal end 202 of the tube 200 (i.e., which remains external to the patient P, even when in its final position) or by pulling the tube 200 out from the circumferential gap of the second channel 140 (i.e., when present), for example once the intubation device 100 has been completely removed from the patient P but remains in contact with the endotracheal tube 200 (e.g., contact between the proximal end of the tube 200 and the distal end of the second channel 140).

### First Channel

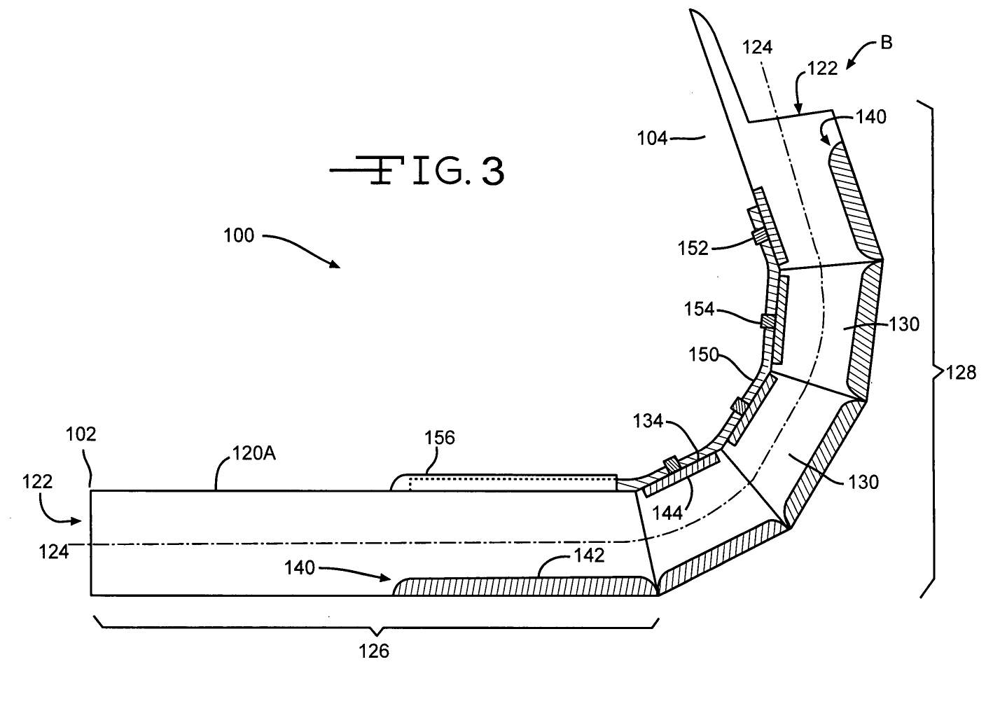

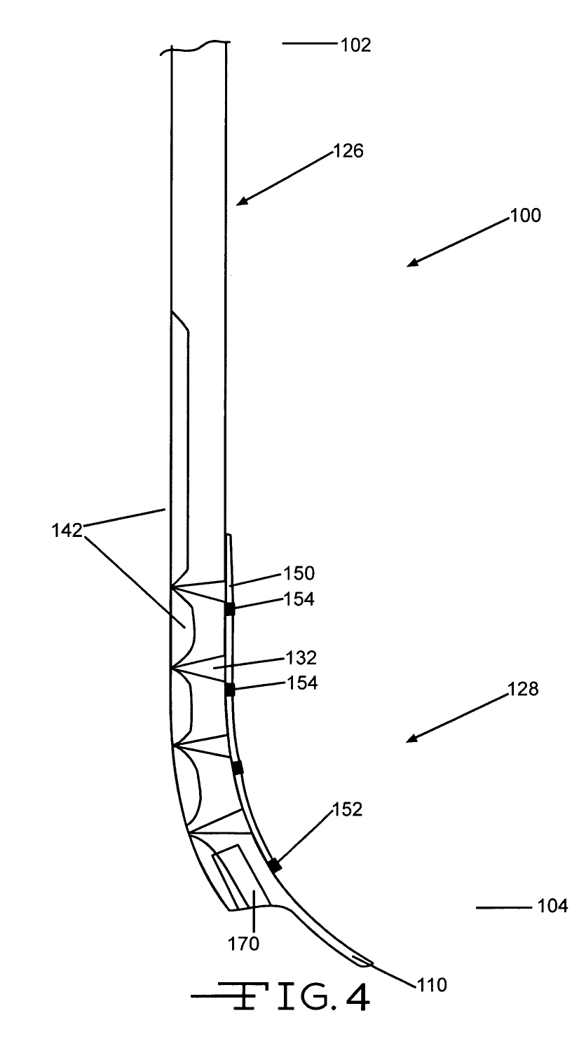

With specific reference to FIGS. 1-8, the channel element 120 that defines the first channel 122 is shown in various views. The channel element 120 has a proximal end 102 (i.e., proximate to the physician M and gripping means 180, if present) and a distal end 104 (i.e., for insertion to the patient P). The channel element 120 generally includes (i) a rigid channel portion 126 at the proximal end 102 of the channel element 120 and (ii) a curveable channel portion 128 at the distal end of 104 the channel element 120. The rigid channel portion 126 and the curveable channel portion 128 generally have a hollow construction (e.g., with interior walls/surfaces and exterior walls/surfaces, not separately illustrated) and are operatively connected to each other so that their hollow interiors define the first channel 122. The first channel 122 extends from the proximal end 102 to the distal end 104 and defines a centerline direction 124 therebetween. The centerline direction 124 essentially follows the contour of the first channel 122 in its current position/degree of articulation. As shown in FIGS. 1 and 2, the centerline direction 124 can be a straight line when the device 100 is in its first relaxed position A (e.g., when the first relaxed position A has essentially no curvature). As shown in FIG. 3, the centerline direction 124 is straight in the rigid channel portion 126 (i.e., which is straight in the illustrated embodiment), but the centerline direction 124 curves and adopts the local curvature of the curveable channel portion 128 when the device 100 is in the second curved position B. The rigid channel portion 126, the curveable channel portion 128, and the first channel 122 are illustrated with a generally rectangular cross section (e.g., normal to the centerline direction 124), but the cross section generally can have any desirable shape (e.g., a circular shape). In an embodiment, the intubation device 100 can include a protrusion 110 extending in the centerline direction 124 from the distal end 104 of the curveable channel portion 126. The protrusion 110 lifts the epiglottis of the patient P when the endotracheal tube 200 is inserted into the patient's trachea T and prevents forward soft tissue within the patient P from contacting the endotracheal tube 200 and undesirably displacing the tube 200 in the longitudinal direction during insertion.

As described in more detail below, the curveable channel portion 128 and the second channel 140 are together continuously and reversibly moveable between the first relaxed position A and the second curved position B upon actuation of the articulating means 160. Thus, the intubation device 100 is generally capable of assuming any configuration intermediate between the two extremes shown in FIGS. 1 and 3 (i.e., relaxed position A and curved position B, respectively) with the appropriate application or release of pressure from the articulating means 160.

The figures generally illustrate an embodiment in which the curveable channel portion 128 is formed from one or more (e.g., a plurality) curveable channel elements 130. The curveable channel elements 130 are a series of channel structures that are serially interconnected to each other and the rigid channel portion 126. The curveable channel elements 130 generally have sidewalls (e.g., as illustrated), but have openings in the longitudinal direction to define the first channel 122 and to permit the pass-through of structure related to the articulating means 160 and/or any sensor means 170 that is present. The channel elements 130 are interconnected at a common edge/location and are disconnected at one or more other edges/other locations. The combination of free and constrained/connected edges between adjacent channel elements 130 permits movement (e.g., curvature) of the curveable channel portion 130. As illustrated, one curveable channel element 130 is connected at its proximal end to the distal end of the rigid channel portion 126 and the other curveable channel elements 130 are connected at the proximal end thereof to the distal end of an adjacent curveable channel element 130. The non-connected portions of the curveable channel elements 130 define a gap (or gaps) 132 on a curveable side 134 of the curveable channel portion 128. Each gap 132 is located between the curveable channel element 130 and (i) the rigid channel portion 126 or (ii) an adjacent curveable channel element 130.

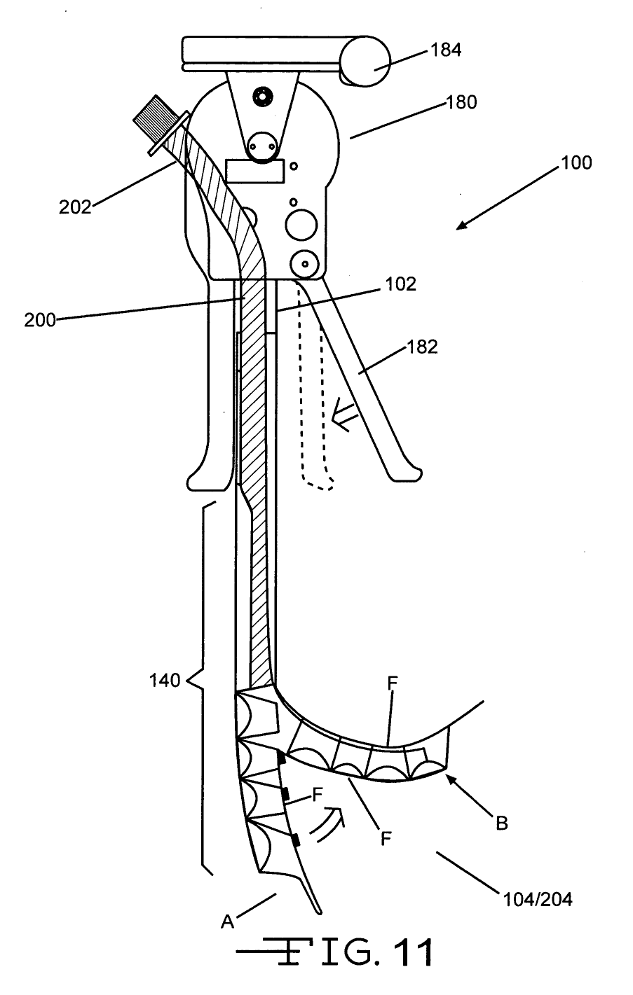

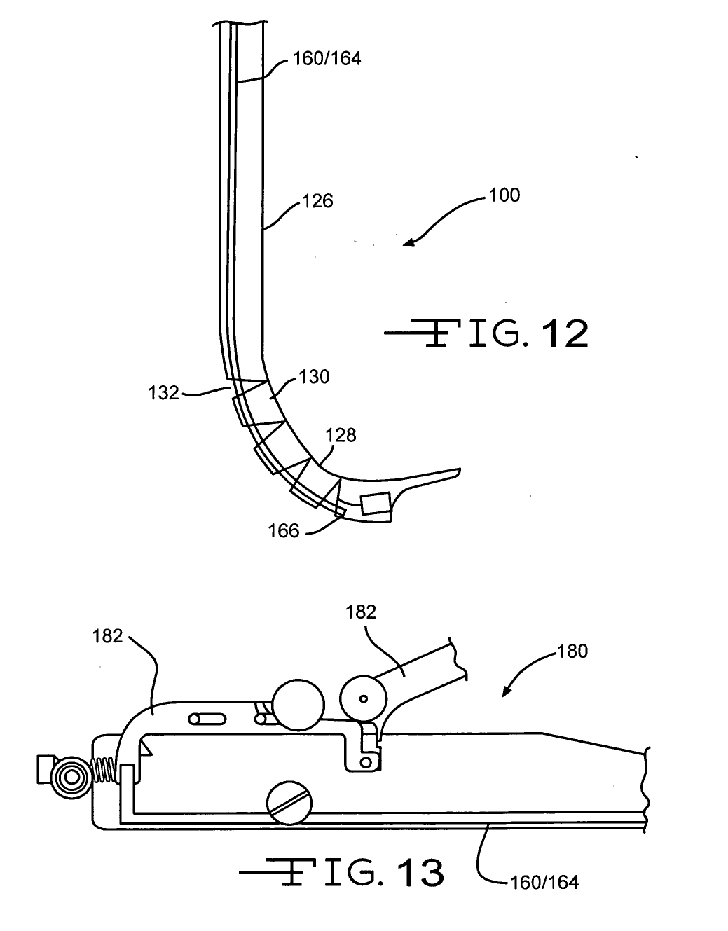

The gap 132 changes in its extent as the intubation device 100 moves between the first relaxed position A and the second curved position B. The curveable side 134 is generally defined as the side or sides of the curveable channel portion 128 where the gaps 132 are located and where the gaps 132 change in size during the articulation of the device 100. In the embodiment illustrated in FIGS. 1-8 (e.g., having a control wire 162 as the articulating means 160), the gaps 132 are present along the sides and top of the curveable channel portion 128. In this embodiment, each gap 132 is at its maximum extent when the curveable channel portion 128 and the second channel 140 are in the first relaxed position A, and each gap 132 is closed (as illustrated) or at its minimum extent (not shown) when the curveable channel portion 128 and the second channel 140 are in the second curved position B. In the embodiment illustrated in FIGS. 12-13 (e.g., having a push rod 164 as the articulating means 160), the gaps 132 are present along the sides and bottom of the curveable channel portion 128. In this embodiment, each gap 132 is present but at its minimum extent when the curveable channel portion 128 and the second channel 140 are in the first relaxed position A, and each gap 132 widens to its maximum extent (as illustrated in FIG. 12) when the curveable channel portion 128 and the second channel 140 are in the second curved position B.

FIGS. 1-8, 12, and 13 illustrate a particular embodiment of the curveable channel elements 130 in which (i) each curveable channel element 130 is hingedly connected (e.g., via a hinge 136A or other suitable rotatable connection means) along a first edge 136 of the curveable channel element 130 to the rigid channel portion 126 (e.g., the leftmost illustrated element 130) or an adjacent curveable channel element 130 (e.g., the other illustrated elements 130), and (ii) the gap 132 of each curveable channel element 132 is defined by a second, opposing edge 138 of the curveable channel element 130. As shown, the channel element 130 has a trapezoidal cross section along the centerline direction 124 such that the non-parallel sides of the trapezoidal cross section define the gaps 132 in the curveable channel portion 128. The trapezoidal cross section can have a trapezoidal frame element that defines an open face that allows access to and cleaning of the first channel 120 interior 120B. In the illustrated embodiment, the gap or gaps 132 has/have a wedge shape between each curveable channel element 130 and the rigid channel portion 126 or its adjacent curveable channel element 130. However, any suitable gap shape is possible, for example including a slit shape (not shown) between each curveable channel element 130 and the rigid channel portion 126 or its adjacent curveable channel element 130. In another embodiment, adjacent channel elements 130 can be interconnected along or near the centerline of the first channel 122 (e.g., along the centerline direction 124), thus creating gaps 132 on multiple sides of the curveable channel portion 126 (e.g., as disclosed in U.S. Publication No. 2010/0095969 to Schwartz et al., incorporated herein by reference).

The channel element 120 and its components (e.g., the rigid channel portion 126 and the curveable channel portion 128) can be fabricated from any biocompatible metallic or plastic material. In an embodiment, the rigid channel portion 126 and the curveable channel portion 128 both are formed from rigid materials (e.g., stainless steel or a shape memory alloy (SMA) such as a nitinol nickel-titanium alloy), in which case the curveable channel portion 128 is formed from one or more components that are flexibly connected to each other or the rigid channel portion (e.g., the curveable channel elements 130 described above). Alternatively, the curveable channel portion 128 can be formed from an integral, flexible material such as a silicone or other plastic tube (e.g., having a circular, rectangular, or other cross section).

### Second Channel

The second channel 140 is adjacent the first channel 122 and extends in the centerline direction 124 along at least a segment of the rigid channel portion 126 and at least a segment of the curveable channel portion 128. The centerline second channel 140 defines a placement direction (or axis; related to the direction/orientation of the endotracheal tube 200 as it is threaded through the second channel 140 and into the patient P) 146 that generally runs parallel to the centerline direction 124 such that the placement direction 146 has the same or similar curvature to that of the centerline direction 124. The second channel 140 exerts a retaining force F on the endotracheal tube 200 when the tube 200 is present in the second channel 140 and the second channel 140 is in the curved position B, in particular when the diameter/width of the tube 200 is selected to correspond to the diameter/width of the second channel 140 or vice versa. For example, the retaining force F can be a frictional force between an interior surface 140B of the second channel 140 and an exterior surface 200A of the endotracheal tube 200 when the diameter/width of the endotracheal tube 200 is sized correspondingly to (e.g., slightly less than) the cross sectional size of the second channel 140. The retaining force F relaxes to release the endotracheal tube 200 as the second channel 140 moves away from the curved position B and toward the relaxed position A. The retaining force F need not be eliminated as the degree of actuation lessens (e.g., some residual retaining or frictional force F can be present in the relaxed position A), but the retaining force F suitably is reduced sufficiently to permit the withdrawal of the intubation device 100 from the patient P once the endotracheal tube 200 is in place.

As illustrated, the second channel 140 is partially open around its circumference and along its length in the centerline direction 124 (or the placement direction 146). The partially open structure permits access to the second channel interior 1408 other than through the proximal and distal ends of the second channel 140. Specifically, the partially open structure provides sufficient retaining structure to hold the endotracheal tube 200 in place during an intubation process, but the circumferential gap can permit a flexible tube 200 to be removed post-intubation (e.g., laterally removed via the circumferential gap either instead of complete or in addition to partial longitudinal removal through the distal end of the second channel 140). In another embodiment (not shown), however, the second channel 140 can be completely enclosed in the circumferential direction such that the endotracheal tube 200 is inserted into, advanced through, and eventually removed from the intubation device 100 via the open proximal and distal ends of the second channel 140. In the illustrated embodiment, the second channel 140 is defined by one or more retaining lips 142 and one or more overhang structures 144, both of which generally extend outwardly from the first channel 122 (e.g., as integral structures/extensions of the outside wall 120A of the channel element 120 and its component rigid and curveable channel portions 126, 128) and define a slit/gap 145 therebetween that permits circumferential access to the second channel 140 (i.e., as compared to longitudinal access to the second channel 140 at the distal and proximal ends thereof). The lips 142 and overhangs 144 extend in a direction that is substantially normal to the centerline direction 124. The retaining lips 142 have a curved surface (e.g., that extends normally outward and then curves upwardly or otherwise toward the centerline of the second channel 140) that is sized and located to retain an endotracheal tube 200 within the second channel 140. Alternatively, the retaining lips 142 could have any other suitable cross sectional shape to retain the tube 200 and prevent/limit the lateral movement of the tube 200 during intubation (e.g., a rectangular or other non-linear bend shape that need not have curved arc segment). In another embodiment (not shown), the overhangs 144 can similarly have a curved or other cross sectional shape to retain and limit the lateral movement of the tube 200, either in addition to or in place of such a structure for the lips 142.

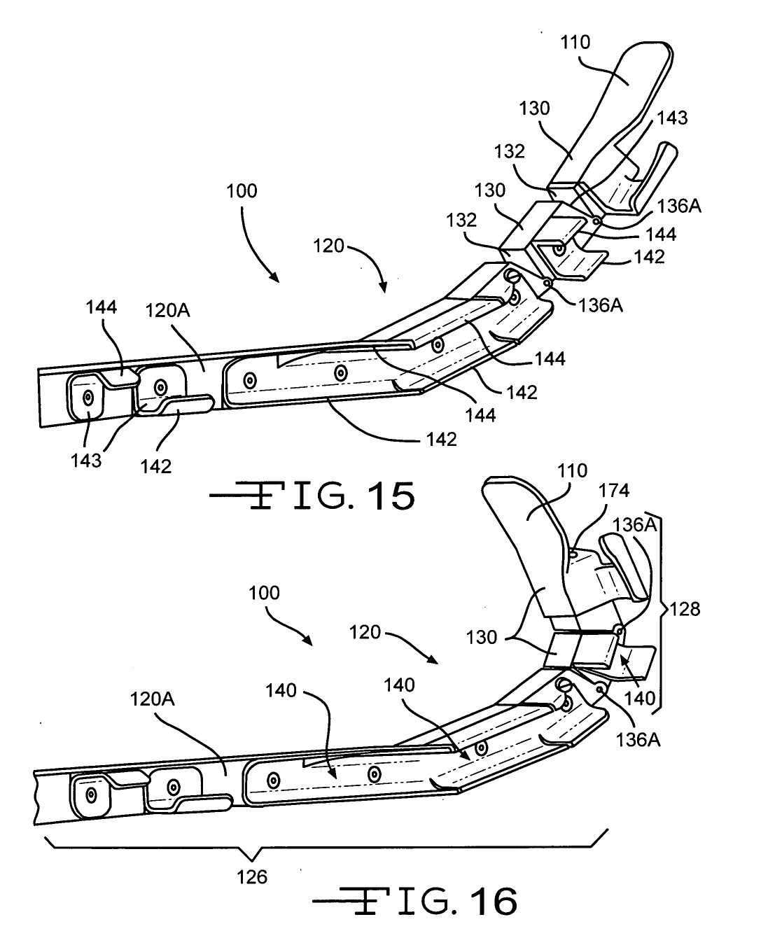

As illustrated in FIGS. 1-4, the lips 142 and overhangs 144 are integral structures extending from the outside wall 120A of the channel element 120 and its component rigid and curveable channel portions 126, 128. In another embodiment illustrated in FIGS. 15 and 16, the lips 142 and overhangs 144 and can be defined by one or more (e.g., a plurality) of clips 143 that are attached to the outside wall 120A of the channel element 120 and that have outwardly extending clip fingers. The outwardly extending clip fingers correspond to the lips 142 and overhangs 144 in the drawings and together define the second channel 140. As shown, the clips 143 can variously include either or both of the lips 142 and overhangs 144 as clip fingers (e.g., only one overhang 144 or only one lip 142 as shown in the leftmost clips 143 of FIGS. 15 and 16). FIG. 15 further illustrates an embodiment in which the intubation device 100 is partially curved in its relaxed state. This facilitates the transition from the relaxed state (FIG. 15; low degree of curvature) to a fully articulated state (FIG. 16; higher degree of curvature) with relatively fewer curveable channel elements 130 (e.g., two curveable channel elements 130 as shown in FIGS. 15 and 16 as compared four curveable channel elements 130 as shown in FIGS. 1-5, including the terminal element 130 with the protrusion 110). Thus, the intubation device 100 can have a straight or substantially straight configuration in the first relaxed position A in some embodiments (e.g., as illustrated in FIGS. 1 and 2), such as when an angle/degree of curvature between the distal end of the centerline direction 124 and the proximal end of the centerline direction (e.g., corresponding to a longitudinal axis of the distal-most channel element 130 and a longitudinal axis of the rigid channel portion 126) ranges between 0° (i.e., straight) and 5°, 10°, or 20°. Conversely, the intubation device 100 can have a slightly curved configuration in the first relaxed position A in other embodiments (e.g., as illustrated in FIG. 15), such as when the angle/degree of curvature between the distal end of the centerline direction 124 and the proximal end of the centerline direction is at least 5°, 10°, or 20° and/or up to 20°, 40°, or 60°. The interior of the channel element 120 in the embodiment of FIGS. 15 and 16 is substantially the same as described with respect to the foregoing figures (e.g., as illustrated in FIG. 8 and including an articulating means 160/control wire 162 and a conductor 172 for sensor 170).

While the second channel 140 can extend along the entire length of the intubation device 100, the second channel 140 as illustrated extends along only a distal segment of the rigid channel portion 126. Such a configuration provides sufficient structure to retain the endotracheal tube 200 during insertion, but facilitates the disengagement of the tube 200 from the device 100 after insertion.

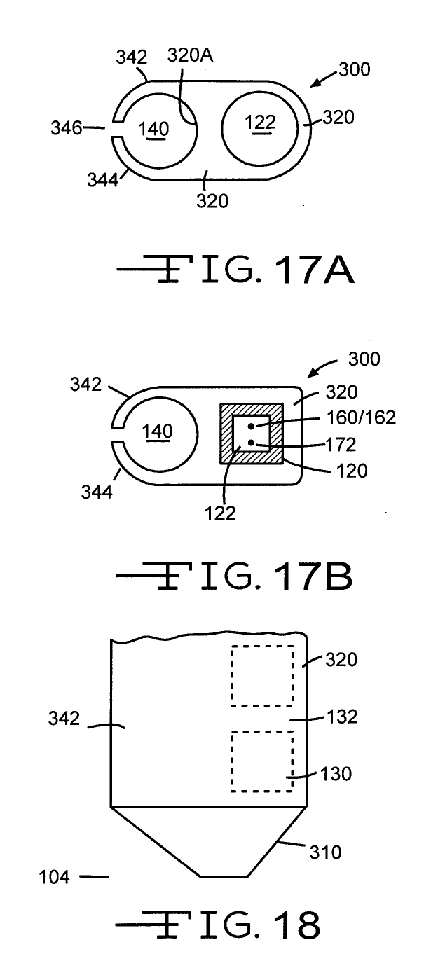

FIGS. 17 and 18 illustrate an embodiment in which the second channel 140 is defined by a flexible sheath 300 that encases the articulating channel element 120. The flexible sheath 300 can be disposable (i.e., discarded at the end of an intubation process with a new sheath 300 being placed over/around the channel element 120 before a new procedure) and is suitably formed from a flexible polymer material such as silicone, latex, etc. As shown in FIG. 17A, the sheath 300 can be formed from flexible plastic tubing defining two side-by-side channels (e.g., sized to correspond to the first and second channels 122, 140 as shown and to accommodate the channel element 120 and the endotracheal tube 200, respectively). A channel wall 320 portion of the sheath 300 generally defines an enclosed channel section that encases the channel element 120 once inserted into the sheath 300. The sheath 300 can encase substantially the entire length of the channel element 120 (i.e., the rigid and curveable channel portions 126, 128) or a portion thereof (e.g., all or a portion of the rigid channel portion 126 and/or all or a portion of the curveable channel portion 128). Two retaining lips 342, 344 (e.g., or other retaining surfaces, one of which can be an outer surface 320A of the channel wall 320) attached or otherwise protruding from the channel wall 320 (e.g., extending outwardly generally in a direction that is substantially normal to the centerline direction 124). The lips 342, 344 suitably are integrally formed with the sheath 300, For example, the tubular channel sized for the endotracheal tube 200 can have a slit or otherwise define a gap 346 along the longitudinal length of the sheath 300 and at any suitable circumferential position to permit insertion of the endotracheal tube 200 into the second channel 140 to be retained by the lips 342, 344.

FIG. 17B illustrates the flexible sheath 300 with the channel element 120 inserted therein (e.g., where a generally round channel 122 of the flexible sheath 300 deforms to accommodate a generally square channel element 120). At this point, the endotracheal tube 200 (not shown) can be inserted into the second channel 140 to perform an intubation process. The channel element 120 in FIG. 17B can represent any portion of the element 120 (e.g., the rigid channel portion 126, the curveable channel portion 128, the curveable channel element 130), depending on the particular longitudinal position of the cross section. FIG. 18 illustrates a top view of the distal end 104 of the flexible sheath 300. Suitably, a plastic tip 310 (e.g., a rigid plastic material) can extend from the flexible sheath 300 (e.g., as an alternative to the protrusion 110 at the distal end 104 of the channel element 120). The dashed lines in FIG. 18 illustrate the interior position of the curveable channel elements 130 and the corresponding gap 132 for an inserted channel element 120.

### Articulating Means

The articulating means 160 of the intubation device 100 extends through the first channel 122 between the proximal end 102 and the distal end 104. The articulating means 160 is operatively connected to the curveable channel portion 128 for articulation, for example being anchored to an interior wall 120B of the first channel 122/curveable channel portion 128 at or near the distal end 104 (e.g., at an anchor point 166 using an adhesive, pin, screw, etc. or other suitable fastening means). The structure of the articulating means 160 is not particularly limited, but two suitable options include a control wire 162 (shown in FIGS. 1-8) or a push rod 164 (shown in FIGS. 12-13), either of which can be anchored to the interior wall 120B of the first channel 122 at or near the distal end 104 thereof. The articulating means 160 in any of its forms can be isolated from other components within the first channel 122 and from elements from the external environment (e.g., within the intubation passageways of a patient P), for example using a polymer wrap/boot that can isolate individual components or the entire channel.

Actuation of the articulating means 160 causes the intubation device 100 (i.e., and its component first and second channels 122, 140) to move incrementally between the first relaxed position A and the second curved position B, and vice versa (FIG. 11). For example, application of tension (or pulling force) to the proximal end of the control wire 162 causes the device 100 to move away from the relaxed position A toward the curved position B, while relaxation or removal of the tension causes the device 100 to move back towards the relaxed position A (FIGS. 1 and 3). Similarly, application of a pushing force to the proximal end of the push rod 164 causes the device 100 to move away from the relaxed position A toward the curved position B, while relaxation or removal of the pushing force causes the device 100 to move back towards the relaxed position A (FIG. 12).

### Gap Cover Plate

In the illustrated embodiment, the endotracheal intubation device 100 can include a flexible gap cover plate 150 on an outside wall 120A of the channel element 120 adjacent the gap or gaps 132 (e.g., when such gaps are present based on a configuration including the curveable channel elements 130). The gap cover plate 150 covers at least a portion of the gap or gaps 132 and at least partially shields the first channel 122 interior from the external environment (e.g., internal patient intubation passageways). The gap cover plate 150 is anchored to the channel element 120 in one location, for example to the rigid channel portion 126 or to the curveable channel portion 128 (e.g., via any suitable mechanical or adhesive means, shown by an anchor point 152 in the figures). The gap cover plate 150 also is slidably retained at one or more locations (e.g., illustrated by one or more retaining bands 154 defining an exterior channel segment on an outer surface of the first channel 122 and/or flexible channel portion 128) on the channel element 120 so that the plate 150 can slide/move in the centerline direction 124. The gap cover plate 150 is flexible (e.g., formed from a thin metallic or plastic material) so that it conforms to the shape of the curveable channel portion 128 as the curveable channel portion 128 moves between the first relaxed position A and the second curved position B. The intubation device 100 additionally can include a retaining sleeve 156 at the distal end of the rigid channel portion (as shown), at the proximal end of the curveable channel portion (not shown), or at both locations (not shown). The retaining sleeve 156 is positioned and sized to enclose a freely moving/sliding end of the gap cover plate 150 as the curveable channel portion 128 moves between the first relaxed position A and the second curved position B, thus preventing the freely moving end from disengaging from the device 100 outer surface and contacting or damaging an internal portion of the patient's intubation passageways.

### Additional Components

The intubation device 100 can include other ancillary components useful for an intubation process, for example including a sensor means 170 and a gripping means 180, both of which are shown in the figures. Suitable sensor means 170 and gripping means 180 are generally described below; other suitable structures may be found in the related patents and patent applications referenced above in the Summary section.

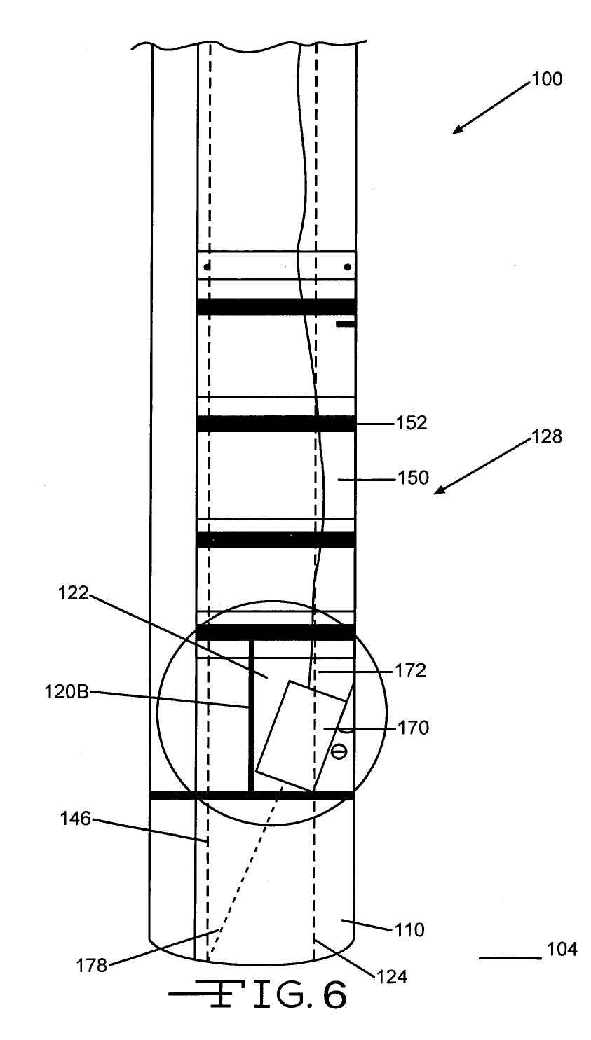

The sensor means 170 generally includes any structure located on or within the device 100 that provides information/feedback (e.g., visual) to the physician M during an intubation process to facilitate the accurate placement of the endotracheal tube 200 within the patient P. In the illustrated embodiment, the sensor means 170 is mounted within the first channel 122 at or near the distal end 104 of the curveable channel portion 128 (e.g., in a terminal curveable channel element 130). The sensor means 170 can include a camera or imaging unit 174 (e.g., a CMOS imager, a CCD imager, an FPA imager, an IR imager, and an ultrasonic imager) to provide visual information to the physician M. Additionally, the sensor means 170 can include an illumination unit 176 (e.g., a LED or other light source) that enhances the imaging ability of the imaging unit 174. The sensor means 170 is electrically connected to an external power source and/or viewing means 184 (described below) via a conductor 172 that runs through the first channel 122. Similar to the articulating means 160, the conductor 172 can be isolated from other components within the first channel 122 and from elements of the external environment (e.g., within the intubation passageways of a patient P), for example using a polymer wrap/boot that can isolate individual components or the entire channel. As illustrated in FIG. 6, the sensor means 170 generally defines a sensing axis 178 (e.g., the direction of imaging or illumination of the sensor 170) that can be directed toward or otherwise angled relative to a placement axis 146 extending from the distal end of the second channel 140 in the centerline direction 124. As indicated in FIG. 6, the placement axis 146 is generally parallel to (but laterally displaced from) the centerline direction 124 and suitably corresponds to the centerline of the second channel 140 and/or the longitudinal axis of the endotracheal tube 200 when present in the second channel 140. The angle between the sensing axis 178 and the placement axis 146 (e.g., illustrated equivalently as angle θ in FIG. 6 between the sensing axis 178 and the centerline direction 124 and/or the channel element 120 sidewall). Such an orientation helps to ensure optimal information feedback to the physician M during the intubation process.

The gripping means 180 provides a convenient structure for the physician M to hold/grip/direct the intubation device 100 during an intubation process. The gripping means 180 includes an actuating means 182 (e.g., trigger) for the articulation means 160 and facilitates the application or removal of force to the articulation means 160. As illustrated, the intubation device 100 and the gripping means 180 can be assembled into a composite unit in which the proximal end of the rigid channel portion 126 is mounted to the gripping means 160, and the actuating means 182 is operably connected to the proximal end of the articulating means 160. When the sensor means 170 is present, the device 100 can additionally include a viewing means 184 electrically connected to the sensor means 170 through the first channel 122 (e.g., via the conductor 172). The viewing means 184 can include any suitable display (e.g., an LCD display or an OLED display) to display data/images acquired by the imaging unit 174 during intubation. Additionally, the gripping means 180 can incorporate a DC power supply (e.g., an internal battery), for example in the viewing means 184 structure.

Because other modifications and changes varied to fit particular operating requirements and environments will be apparent to those skilled in the art, the disclosure is not considered limited to the examples chosen for purposes of illustration, and covers all changes and modifications which do not constitute departures from the true spirit and scope of this disclosure.

Accordingly, the foregoing description is given for clarity of understanding only, and no unnecessary limitations should be understood therefrom, as modifications within the scope of the disclosure may be apparent to those having ordinary skill in the art.

Throughout the specification, where the compositions, processes, apparatus, or systems are described as including components, steps, or materials, it is contemplated that the compositions, processes, or apparatus can also comprise, consist essentially of, or consist of, any combination of the recited components or materials, unless described otherwise. Component concentrations expressed as a percent are weight-percent (% w/w), unless otherwise noted. Numerical values and ranges can represent the value/range as stated or an approximate value/range (e.g., modified by the term “about”). Combinations of components are contemplated to include homogeneous and/or heterogeneous mixtures, as would be understood by a person of ordinary skill in the art in view of the foregoing disclosure.

What is claimed is:

1. An endotracheal intubation device comprising:
(a) a channel element having a proximal end and a distal end, the channel element comprising
(i) a rigid channel portion at the proximal end of the channel element and
(ii) a curveable channel portion at the distal end of the channel element, the curveable channel portion being operatively connected to the rigid channel portion,
wherein the rigid channel portion and the curveable channel portion together define a first channel that (A) extends from the proximal end to the distal end and (B) defines a centerline direction between the proximal end and the distal end;
(b) an articulating means extending through the first channel between the proximal end and the distal end, the articulating means being operatively connected to the curveable channel portion for articulation; and
(c) a second channel adjacent the first channel and extending in the centerline direction along at least a segment of the rigid channel portion and at least a segment of the curveable channel portion;
wherein:
(i) the curveable channel portion and the second channel are together continuously and reversibly moveable between a first relaxed position and a second curved position upon actuation of the articulating means; and
(ii) the second channel (A) exerts a retaining force on an endotracheal tube when present in the second channel and the second channel is in the curved position and (B) relaxes the retaining force to release the endotracheal tube as the second channel moves from the curved position toward the relaxed position.

2. The endotracheal intubation device of claim 1, wherein:
(i) the curveable channel portion comprises one or more curveable channel elements;
(ii) one curveable channel element is connected at its proximal end to the distal end of the rigid channel portion and the other curveable channel elements, when present, are connected at the proximal end thereof to the distal end of an adjacent curveable channel element;
(iii) each curveable channel element defines a gap on a curveable side of the curveable channel portion, the gap being located between the curveable channel element and (A) the rigid channel portion, (B) an adjacent curveable channel element, or (C) both (A) and (B); and
(iv) the gap changes in its extent as the curveable channel portion and the second channel move between the first relaxed position and the second curved position.

3. The endotracheal intubation device of claim 2, wherein the gap is at its maximum extent when the curveable channel portion and the second channel are in the first relaxed position, and the gap is closed or at its minimum extent when the curveable channel portion and the second channel are in the second curved position.

4. The endotracheal intubation device of claim 2, wherein the gap is at its minimum extent when the curveable channel portion and the second channel are in the first relaxed position, and the gap is at its maximum extent when the curveable channel portion and the second channel are in the second curved position.

5. The endotracheal intubation device of claim 2, wherein:
(i) each curveable channel element is hingedly connected along a first edge of the curveable channel element to the rigid channel portion or an adjacent curveable channel element; and
(ii) the gap of each curveable channel element is defined by a second, opposing edge of the curveable channel element.

6. The endotracheal intubation device of claim 2, wherein the curveable channel element has a rectangular cross section normal to the centerline direction.

7. The endotracheal intubation device of claim 6, wherein the curveable channel element has a trapezoidal cross section along the centerline direction, the non-parallel sides of the trapezoidal cross section defining the gap or gaps in the curveable channel portion.

8. The endotracheal intubation device of claim 7, wherein the trapezoidal cross section comprises a trapezoidal frame element defining an open face that allows access to and cleaning of the first channel interior.

9. The endotracheal intubation device of claim 2, wherein the curveable channel element has a circular cross section normal to the centerline direction.

10. The endotracheal intubation device of claim 2, wherein the gap has a wedge shape between each curveable channel element and the rigid channel portion or its adjacent curveable channel element.

11. The endotracheal intubation device of claim 2, wherein the gap has a slit shape between each curveable channel element and the rigid channel portion or its adjacent curveable channel element.

12. The endotracheal intubation device of claim 2, further comprising a flexible gap cover plate on an outside wall of the channel element adjacent the gap or gaps, wherein:
(i) the gap cover plate covers at least a portion of the gap or gaps and at least partially shields the first channel interior from the external environment;
(ii) the gap cover plate is anchored to the channel element in one location;
(iii) the gap cover plate is slidably retained at one or more locations on the channel element in the centerline direction; and
(iv) the gap cover plate is flexible so that it conforms to the shape of the curveable channel portion as the curveable channel portion moves between the first relaxed position and the second curved position.

13. The endotracheal intubation device of claim 12, wherein the gap cover plate is anchored to one of the curveable channel elements.

14. The endotracheal intubation device of claim 12, wherein the gap cover plate is anchored to the rigid channel portion.

15. The endotracheal intubation device of claim 12, wherein the gap cover plate is slidably retained by the curveable channel element with a retaining band thereon that defines a channel through which the gap cover plate can slide in the centerline direction.

16. The endotracheal intubation device of claim 12, further comprising a retaining sleeve at the distal end of the rigid channel portion, at the proximal end of the curveable channel portion, or both, wherein the retaining sleeve is positioned and sized to enclose a freely moving end of the gap cover plate as the curveable channel portion moves between the first relaxed position and the second curved position.

17. The endotracheal intubation device of claim 1, wherein the second channel is partially open around its circumference and along its length in the centerline direction, the partially open structure permitting access to the second channel interior other than through the proximal and distal ends of the second channel.

18. The endotracheal intubation device of claim 17, wherein:
(i) the second channel is defined by one or more retaining lips and one or more overhang structures, both of which generally extend outwardly from the first channel in a direction that is substantially normal to the centerline direction; and
(ii) the retaining lips comprise a curved surface that is sized and located to retain an endotracheal tube within the second channel.

19. The endotracheal intubation device of claim 17, wherein
(i) the second channel is defined by a first retaining surface and an opposing second retaining surface, each extending from or defined by a flexible sheath encasing at least a portion of the channel element; and
(ii) the first retaining surface and second retaining surface are sized and located to retain an endotracheal tube within the second channel.

20. The endotracheal intubation device of claim 19, wherein the flexible sheath is formed from a flexible polymer.

21. The endotracheal intubation device of claim 1, wherein the curveable channel portion and the second channel are substantially straight in the first relaxed position.

22. The endotracheal intubation device of claim 1, wherein the curveable channel portion and the second channel (i) are partially curved in the first relaxed position and (ii) have a greater degree of curvature in the second curved position.

23. The endotracheal intubation device of claim 1, wherein the second channel extends along only a distal segment of the rigid channel portion.

24. The endotracheal intubation device of claim 1, wherein the retaining force comprises a frictional force between an interior surface of the second channel and an exterior surface of the endotracheal tube, the diameter of the endotracheal tube being sized correspondingly to the cross sectional size of the second channel.

25. The endotracheal intubation device of claim 1, wherein the articulating means is anchored to an interior wall of the first channel at or near the distal end.

26. The endotracheal intubation device of claim 1, wherein the articulating means comprises a control wire that is anchored to an interior wall of the first channel at or near the distal end.

27. The endotracheal intubation device of claim 1, wherein the articulating means comprises a push rod that is anchored to an interior wall of the first channel at or near the distal end.

28. The endotracheal intubation device of claim 1, further comprising a sensor means mounted within the first channel at or near the distal end of the curveable channel portion.

29. The endotracheal intubation device of claim 28, wherein the sensor means comprises an imaging unit selected from the group consisting of a CMOS imager, a CCD imager, an FPA imager, an IR imager, and an ultrasonic imager.

30. The endotracheal intubation device of claim 29, wherein the sensor means further comprises an illumination source to enhance the imaging ability of the imaging unit.

31. The endotracheal intubation device of claim 28, wherein the sensor means has a sensing axis that is directed toward a placement axis extending from the distal end of the second channel in the centerline direction.

32. The endotracheal intubation device of claim 1, further comprising a protrusion extending in the centerline direction from the distal end of the curveable channel portion, the protrusion being operable to lift the epiglottis of a patient when inserted into the trachea of a patient.

33. The endotracheal intubation device of claim 1, further comprising a gripping means that comprises an actuating means for the articulation means, wherein:
(i) the proximal end of the rigid channel portion is mounted to the gripping means; and
(ii) the actuating means is operably connected to the proximal end of the articulating means.

34. The endotracheal intubation device of claim 33, further comprising a sensor means mounted within the first channel at or near the distal end of the curveable channel portion and a viewing means electrically connected to the sensor means through the first channel.

35. The endotracheal intubation device of claim 34, wherein the viewing means comprises DC power supply and a display selected from the group consisting of an LCD display and an OLED display.

36. The endotracheal intubation device of claim 1, wherein the channel element comprises a material selected from the group consisting of stainless steel and a shape memory alloy (SMA).

37. The endotracheal intubation device of claim 1, wherein the channel element comprises a nitinol nickel-titanium alloy.

38. A method of intubating a patient, the method comprising:
(a) providing the endotracheal intubation device according to claim 1;
(b) advancing an endotracheal tube through the second channel of the endotracheal intubation device;
(c) inserting the distal end of the channel element with the endotracheal tube in the second channel into a patient's mouth, wherein the curveable channel portion and the second channel are in the first relaxed position during insertion;
(d) actuating the articulating means to move the curveable channel portion and the second channel toward the second curved position by an amount sufficient to allow the distal end of the channel element and the endotracheal tube to be safely advanced in the throat of the patient;
(e) advancing the distal end of the channel element to a position allowing guidance of the endotracheal tube into the trachea of the patient;
(f) advancing the distal end of the endotracheal tube into the trachea of the patient;
(g) releasing the articulating means, thereby relaxing the curveable channel portion and allowing the curveable channel portion to conform to an interior patient passageway defined by the patient's anatomy; and
(h) removing the endotracheal intubation device from the patient's mouth while holding the endotracheal tube in place.

39. The method of claim 38, wherein:
(i) the retaining force comprises a frictional force between an interior surface of the second channel and an exterior surface of the endotracheal tube, the diameter of the endotracheal tube being sized correspondingly to the cross sectional size of the second channel; and
(ii) the release of the articulating means in part (g) reduces the frictional force, thereby facilitating the removal of the endotracheal intubation device in part (h).
